(19) United States
(12) Patent Application Publication
(10) Pub. No.: US 2007/0095160 A1
(43) Pub. Date: May 3, 2007

(54) Structural assessment and monitoring system and associated method

(75) Inventors: Gary E. Georgeson, Federal Way, WA (US); Marc Matsen, Seattle, WA (US); Bud Westerman, Auburn, WA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The Boeing Company

(21) Appl. No.: 11/266,459

(22) Filed: Nov. 3, 2005

(51) Int. Cl. G01N 33/00

(52) U.S. Cl. 73/866

(57) ABSTRACT

A system and method for assessing and monitoring a structure are provided. The system includes at least one radio frequency identification tag, and at least one non-destructive inspection sensor for acquiring data indicative of the structure and providing the acquired data to the radio frequency identification tag. The system could also include a data acquisition system capable of wirelessly communicating with the radio frequency identification tag and providing information indicative of a defect in the structure based on the data acquired by the non-destructive inspection sensor.

### BACKGROUND OF THE INVENTION

1) Field of the Invention

Embodiments of the present invention relate to a structural assessment and monitoring system and, more particularly, to a structural assessment and monitoring system utilizing wireless communication to inspect a structure.

2) Description of Related Art

Non-destructive inspection (NDI) of structures involves thoroughly examining a structure without harming the structure or requiring its significant disassembly. Non-destructive inspection is typically preferred to avoid the schedule, labor, and costs associated with removal of a part for inspection, as well as avoidance of the potential for inducing damage into the structure. Non-destructive inspection is advantageous for many applications in which a thorough inspection of the exterior and/or interior of a structure is required. For example, non-destructive inspection is commonly used in the aircraft industry to inspect aircraft structures for any type of internal or external damage to or defects (flaws) in the structure. Inspection may be performed during manufacturing or after the completed structure has been put into service, including field testing, to validate the integrity and fitness of the structure. In the field, access to interior surfaces of the structure is often restricted, requiring disassembly of the structure, introducing additional flow time and labor costs.

Among the structures that are routinely non-destructively tested are composite structures, such as composite sandwich structures and other adhesive bonded panels and assemblies and structures with contoured surfaces. These composite structures, and a shift toward lightweight composite and bonded materials such as using graphite materials, dictate that devices and processes are available to ensure structural integrity, production quality, and life-cycle support for safe and reliable use. As such, it is frequently desirable to inspect structures to identify any defects, such as cracks, discontinuities, voids, or porosity, which could adversely affect the performance of the structure. For example, typical defects in composite sandwich structures, generally made of one or more layers of lightweight honeycomb or foam core material with composite or metal skins bonded to each side of the core, include disbonds which occur at the interfaces between the core and the skin or between the core and a buried septum.

Various types of sensors may be used to perform non-destructive inspection. One or more sensors may move over the portion of the structure to be examined, and receive data regarding the structure. For example, a pulse-echo (PE), through transmission (TT), or shear wave sensor may be used to obtain ultrasonic data, such as for thickness gauging, detection of laminar defects and porosity, and/or crack detection in the structure. Resonance, pulse-echo, or mechanical impedance sensors are typically used to provide indications of voids or porosity, such as in adhesive bondlines of the structure. High resolution inspection of aircraft structure is commonly performed using ultrasonic testing (UT) to provide a plan view image of the part or structure under inspection. Data acquired by sensors is typically processed and then presented to a user via a display as an image of the inspected structure. To increase the rate at which the inspection of a structure is conducted, a scanning system may include arrays of inspection sensors, i.e., arrays of transmitters and/or detectors. Non-destructive inspection may be performed manually by technicians who typically move an appropriate sensor over the structure, by semi-automated inspection systems (e.g., the Mobile Automated Scanner (MAUS®) system), and by automated inspection systems (e.g., Automated Ultrasonic Scanning System (AUSS®) system) that have also been developed.

New aircraft structures comprised of composites, multi-functional systems, and complex geometries create a maintenance burden for aircraft inspection. The maintenance tools, procedures, and practices used on metallic aircraft are generally not compatible or cost effective with next generation aircraft structures. New aircraft structures will most likely be a mix of bonded and bolted laminates with a variety of metallic and composite substructures. Removing panels to gain access to structural components will be more difficult with these new integrated structural systems.

Since composite materials can often hide a defect, a detection system is needed to promote user confidence and to reduce the impact of additional undetected damage growth. The ability to detect flaws, monitor anomalies, or predict damage is dependant on the system and sensors used by inspectors. Current and planned structural health monitoring (“SHM”) systems require a network of sensors, wires, and data ports that may be heavy, bulky, unreliable, and costly. Using existing NDI equipment and processes presents a solution that is costly and time-consuming. In particular, for in-service inspection NDI sensors are generally placed by hand onto the structure under inspection by an inspector, who is unable to easily access all locations that require inspection. Inspection of some areas of an aircraft can be time-consuming and costly because of their locations.

General techniques have been developed to transfer data wirelessly to and from devices located on a structure. For example, U.S. Pat. No. 6,859,757 to Muehl et al. discloses a method for tagging an article, such as an aircraft, with maintenance related information. In particular Muehl discloses using tags, such as radio frequency identification (“RFID”) tags, that are associated with respective components on the aircraft (e.g., body frame or engine). The tags can store information relating to the operation, maintenance, repair, replacement, and technical characteristics of the respective components that each tag is associated with. Moreover, the tag may store information provided by previous non-destructive evaluations of the integrity of a component of the aircraft. An interrogator, such as an optical scanner, may be utilized to read data from the tags or write data to the tags using various techniques. Despite the improvements provided by Muehl, there is a need for an inspection system that includes tags that are capable of not only storing a variety of information that can be accessed and updated via wireless communications, but also performing NDI on the structure.

It would therefore be advantageous to provide a system that is capable of wirelessly inspecting a structure. In addition, it would be advantageous to provide a system that is capable of inspecting structures effectively and efficiently. Furthermore, it would be advantageous to provide a system that is economical to manufacture and use.

### BRIEF SUMMARY OF THE INVENTION

Embodiments of the invention address the above needs and achieve other advantages by providing a structural assessment and monitoring system that is capable of wirelessly monitoring NDI sensors to provide information indicative of a defect in the structure, as well as other information indicative of the structure. In particular, for in-service inspection, NDI sensors are integrated with wireless devices, such as RFID tags, such that the NDI sensors may acquire data and transfer data to a data acquisition system via wireless communications. Thus, data may be acquired from various locations on a structure that would typically be difficult or time consuming to access with conventional inspection techniques to assess the structural integrity of the structure.

In one embodiment of the present invention, a system for assessing and monitoring a structure is provided. The system includes at least one radio frequency identification tag, and at least one non-destructive inspection sensor that is capable of acquiring data indicative of the structure and providing the data to the radio frequency identification tag. The system could also include a data acquisition system that is capable of wirelessly communicating with the radio frequency identification tag and providing information indicative of a defect in the structure based on the data acquired by the non-destructive inspection sensor.

In various aspects of the system, the radio frequency identification tag can be integrated with the non-destructive inspection sensor, such as a piezoelectric sensor. The radio frequency identification tag and non-destructive sensor may be attached to, or embedded within, the structure, and could alternatively be carried by an appliqué or a repair patch. The radio frequency identification tag and/or non-destructive inspection sensor can store the data acquired by the non-destructive inspection sensor and/or the information provided by the data acquisition system. The system could also include a non-conductive standoff or a high permeability material backing positioned between the radio frequency identification tag and the structure, where the structure could be a metallic material. Moreover, the system could further include one or more power sources for powering at least one of the radio frequency identification tag and the non-destructive inspection sensor.

Another aspect of the present invention provides a wireless device for assessing and monitoring a structure. The wireless device includes at least one radio frequency identification tag capable of acquiring data indicative of a structure, wherein the radio frequency identification tag is also capable of wirelessly communicating with a data acquisition system. In variations of the wireless device, the radio frequency identification tag is attached to, or embedded within, the structure. The radio frequency identification tag could be carried by an appliqué or a repair patch. In addition, the radio frequency identification tag may be capable of storing the acquired data, as well as communicating the acquired data to the data acquisition system. At least one characteristic of the radio frequency identification tag may be modified (e.g., a deformed antenna) to acquire data indicative of the structure.

A further aspect of the present invention provides a method for assessing and monitoring a structure. The method includes acquiring data indicative of the structure with at least one non-destructive inspection sensor, and providing the data to at least one wireless communication device carried by the structure. The method also includes wirelessly transmitting the data from the at least one wireless communication device, and characterizing a defect within the structure based on the data acquired by the at least one non-destructive inspection sensor and transmitted by the at least one wireless communication device.

Aspects of the method include positioning the wireless communication device and non-destructive inspection sensor within, or adjacent to, the structure, such as by embedding the at least one wireless communication device and non-destructive inspection sensor within the structure, an appliqué, or a repair patch. In addition, the method could include positioning a non-conductive spacer or a high permeability backing material between the wireless communication device and the structure. The method may further include storing the acquired data or information indicative of the defect with the at least one wireless communication device or non-destructive inspection sensor.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a plan view of an inspection system for monitoring and assessing a structure according to one embodiment of the present invention;

FIGS. 2A-2B are cross-sectional views of a wireless device according to one embodiment of the present invention;

FIG. 3 is an elevational view of an inspection system for monitoring and assessing an aircraft according to another embodiment of the present invention; and

FIG. 4 is a flowchart of a method for monitoring and assessing a structure according to one embodiment of the present invention.

### DETAILED DESCRIPTION OF THE INVENTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Referring now to the drawings and, in particular to FIG. 1, there is shown an inspection system 10 for monitoring and assessing a structure. The inspection system 10 includes a plurality of wireless devices 14 positioned on a structure 12. The wireless devices 14 are capable of acquiring data indicative of the structure and wirelessly communicating with a data acquisition system 16. The data acquisition system 16 is capable of generating information indicative of a defect in the structure based on the data acquired by the wireless devices. Thus, the structure 12 is capable of being at least periodically, or otherwise repeatedly, monitored so that the structural integrity of the structure may be assessed by identifying any defects in the structure.

The inspection system 10 could be used to inspect any number of structures 12 in a variety of industries where detection of flaws or defects in the structure is required, such as in the aircraft, automotive, marine, or construction industries. The wireless devices 14 are capable of detecting any number of flaws or defects within or along the surface of the structure, such as impact damage (e.g., delaminations and matrix cracking), disbonds (e.g., airframe/reinforcing members or honeycomb composites), discontinuities, voids, or porosity, which could adversely affect the performance of the structure. In addition, the wireless devices 14 could be utilized for various other purposes, such as for storing various types of information, such as data acquired by the wireless devices or provided by the data acquisition system 16, as will be explained in further detail below. Furthermore, the inspection system 10 can be used with additional inspection systems. For example, the inspection system 10 could be used to monitor and assess only those locations on the structure 12 that are not readily accessible, while other conventional inspection techniques could be used to inspect other locations on the structure.

The term “structure” is not meant to be limiting, as the inspection system 10 could be used to inspect any number of parts or structures of different shapes and sizes, such as machined forgings, castings, pipes, or composite panels or parts. The inspection could be performed on newly manufactured structures or existing structures that are being inspected for preventative maintenance purposes. Further, the structure could be any number of materials. For example, the structure could be a metallic material, such as aluminum, or a composite material, such as graphite-epoxy. Moreover, the structure could be an aircraft, such as the Boeing 787, where a substantial portion of the aircraft structure is a composite material (e.g., the fuselage and wings).

Furthermore, the term “wireless device” is not meant to be limiting, as the wireless device could be any device capable of acquiring data indicative of the structure and transferring the data to a data acquisition system via wireless communications. For example, the wireless device is capable of utilizing wireless technology, such as radio frequency emissions (e.g., via broadband, WiFi, Bluetooth®, etc. communication) or other wireless techniques (e.g., via infrared communication) to communicate with the data acquisition system 16, as will be explained in greater detail below.

The wireless devices 14 generally include a wireless communication device, such as a radio frequency identification (“RFID”) tag 18 integrated with, or otherwise in communication with, a non-destructive inspection (“NDI”) sensor 20, as shown in FIG. 1A. Generally, the RFID tag 18 comprises a tag that includes an integrated circuit (IC) chip microprocessor and a resonant circuit formed by a coiled antenna and a capacitor. The RFID tag 18 could be passive, active, read only, and/or read/write. In a passive RFID system, a device, such as a reader, generates a magnetic field at a predetermined frequency. When an RFID tag 18, which usually can be categorized as being either read-only or read/write, is exposed to the magnetic field, a small electric current is induced in the device's resonant circuit. This circuit provides power to the tag, which then modulates the magnetic field in order to transmit information that is pre-programmed on the tag back to the reader at a predetermined frequency. The reader then receives, demodulates, and decodes the signal transmission, and sends the data on to the data acquisition system 16 associated with the inspection system 10 for further processing. An active RFID system operates in much the same way, but in an active system the RFID tag 18 includes its own battery, allowing the tag to transmit data and information, such as at the touch of a button. Read only RFID tags 18 have a permanent memory that may not be modified, while read/write RFID tags are capable of having updated information written to the RFID tag. Furthermore, in an additional embodiment of the present invention, the wireless devices 14 may include one or more power sources. For example, the wireless devices 14 could include one or more thin, flexible batteries, such as those manufactured by Power Paper®, to power the RFID tags 18 and/or sensors 20.

Each of the non-destructive sensors 20 utilized with wireless devices 14 could be any suitable sensor or transducer capable of transmitting and receiving signals, as well as communicating with the RFID tag 18 and/or data acquisition system 16. Each sensor 20 is typically a non-destructive sensor, such that the sensor is capable of inspecting a structure without harming the structure or requiring disassembly of the structure. In the embodiment of the inspection system 10 shown in FIG. 1, each sensor 20 is an ultrasonic sensor, such as a piezo-electric sensor. However, various other sensors may be employed with the inspection system 10 of the present invention, such as pitch-catch, through-transmission, shear-wave, resonance, or mechanical impedance sensors. For instance, pitch-catch sensors could be arranged on the structure 12 such that one sensor could transmit an ultrasonic signal into the structure and be picked up by a receiving sensor.

The sensors 20 are typically utilized to collect data indicative of the structure 12 that may be used by the data acquisition system 16 to characterize a defect in the structure. However, the sensors 20 may also be employed to acquire data for sensing various conditions on the structure. For example, strain gages could be used to determine if composite repairs are required; capacitive or eddy current thickness sensors could be placed in remote regions on the structure 12 where corrosion thinning is present; and remote crack growth could be monitored by a series type strain, eddy current, or crack wire sensor that monitors crack progression.

Thus, the cooperation of the RFID tags 18 and sensors 20 facilitates the collection and communication of data indicative of the structure 12. In particular, the RFID tags 18 and sensors 20 are typically collocated or located proximate to one another, such as that shown in FIG. 1A, to enable communication between one another. Moreover, the RFID tags 18 and sensors 20 may be integrated into a single wireless device such that a NDI sensor could employ a radio frequency transmitter to communicate with the data acquisition system 16. In addition, one or more characteristics of the RFID tag could be changed, such as by deforming the tag's antenna, to change the response of the RFID tag such that the RFID tag may act as a NDI sensor. For example, the RFID tag could be used to acquire data indicative of the structure such as, for example, changes in strain, temperature, or corrosiveness. The RFID tag's deformation would need to be calibrated to the antenna's resonant frequency peak shift to compensate for any thermal effects or any other effects on the tag.

Typically, each RFID tag 18 would be associated with a respective sensor 20, although any number of RFID tags and sensors may cooperate with one another to acquire data indicative of the structure 12. Each sensor 20 would typically acquire data indicative of the structure 12 and communicate the acquired data to a respective RFID tag 20. The sensors 20 and RFID tags 18 can communicate via a direct connection or wirelessly, and each sensor and RFID tag pair may communicate to an additional RFID tag. If the RFID tag 18 and/or sensor 20 are passive, no external electric power is required. However, in some instances, the RFID tag 18 and/or sensor 20 may be active and require an external power source, such as a battery or a power circuit-with-coil that can be inductively charged by placing an inductive probe adjacent to it. Furthermore, the RFID tag 18 and sensor 20 may be any number of sizes and configurations depending on a variety of factors, such as the size, configuration, or type of material of the structure 12, the type of sensor, and/or the type of defect(s) desired to be detected.

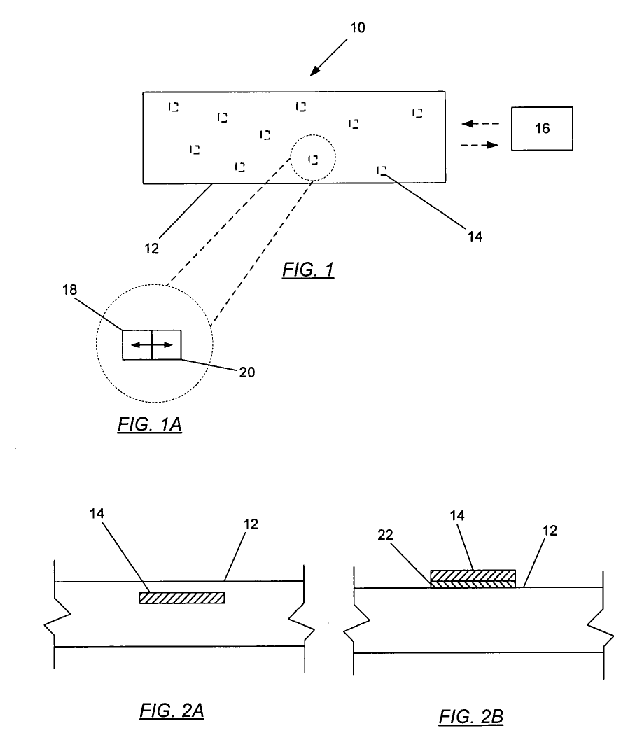

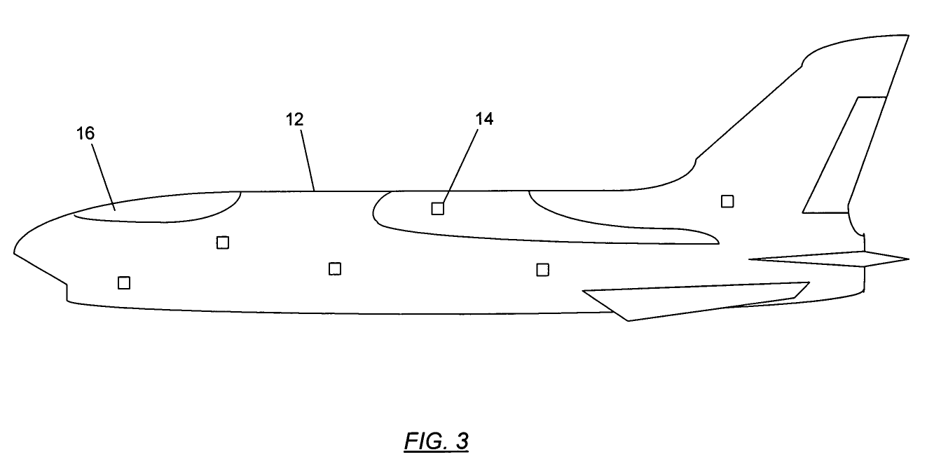

The wireless devices 14 are typically positioned proximate or adjacent to the structure 14. For example, the wireless devices 14 may be embedded within the structure 12, as shown in FIG. 2A, attached to an internal or external surface of the structure, or positioned proximate or adjacent to the structure using other techniques. For instance, the wireless devices 14 may be attached directly to the structure 12 with various fastening techniques, such as adhesives. In addition, the wireless devices 14 could be carried by a repair patch or an appliqué such that the wireless devices acquire data indicative of a specific portion of the structure 12. For example, see U.S. patent application No. ______, entitled “Smart Repair Patch and Associated Method,” respectively, which is filed concurrently herewith, assigned to the present assignee, and incorporated herein by reference, and which provides further details regarding repair patches incorporating wireless devices. Further, the wireless devices 14 could be carried by inspection devices, such as an inspection system for impact-echo testing which is disclosed in further detail in U.S. patent application No. ______, entitled “Non-Destructive Inspection System and Associated Method,” which is assigned to the present assignee and incorporated herein by reference.

In one embodiment of the present invention, the wireless devices 14 can be placed on specific locations on the structure 12 that are expected to encounter a future impact event. An impact near the wireless devices 12 will be picked up by the NDI sensors and translated to an electrical current, which is then stored as a digital value on the RFID tag. In addition, the time that the impact event occurred may be stored by the NDI sensors. In this regard, the NDI sensors may be active and have an external battery source that enables it to acquire data when the impact event occurs. An inspector can check for impact levels near the wireless devices 14. If more than one wireless device 14 is used, and the wireless devices are chronologically interrogated by the data acquisition system 16 or similar device, accurate impact locations can be determined using impact timing and magnitude. This particular application (wireless impact sensing) can be used, for instance, during prototype testing or on aircraft test beds.

Moreover, the RFID tags 18 may become disabled when positioned adjacent to conductive structures due to the fact that the magnetic field generates incident waves that are cancelled when the waves reflect off of the metallic (electrically conductive) surface. Deforming the RFID tag 18 can change the response of the tag and act as a structural inspection sensor. As a result, power to the RFID tag's 18 antenna is negated. In order to reduce power loss to the RFID tag, a standoff 22, such as a non-conductive spacer material (e.g., plexiglas) may be positioned between the RFID tag and the structure 12, as shown in FIG. 2B. Although the standoff 22 could be used on both the interior and exterior of the structure 12, the standoff would typically be used on the interior of the structure due to the thickness of the standoff, which may adversely affect the performance of the structure or physically interfere in some manner if positioned on the exterior of the structure (e.g., the standoff may cause turbulence over portions of an aircraft). In one embodiment of the present invention, the standoff is about 0.125 to 0.25 inches in thickness, although the standoff may be various configurations and sizes if desired.

Furthermore, a high permeability material backing material (e.g., polyvinylidenefluoride (“PVDF”)) may be applied to the RFID tag 18. The backing material has a combination of high magnetic permeability in addition to low electrical conductivity such that the backing material may improve the coupling between the RFID tag 18 and a RFID reader/writer. The backing material is also thin (e.g., less than 0.040 inches) and may be employed to adhere the RFID tag to the structure. Because the backing material is thin, the backing material is capable of being used on both the interior and exterior of the structure 12. An additional option for attaching the RFID tag 18 to a conductive surface is to attach a thin, flexible battery, such as that manufactured by Power Paper®, that will provide sufficient power for the antenna to function in an active mode, and thereby communicate with a wireless device.

The data acquisition system 16 wirelessly communicates with the wireless devices 14. In particular, the data acquisition system 16 is capable of both interrogating the wireless devices 14 to cause the sensors 20 to acquire data indicative of the structure (see FIG. 4, block 24) and wirelessly transferring/receiving data to/from the sensors via the RFID tags 18 (see FIG. 4, blocks 26 and 28). As such, no wiring is necessary to initiate interrogation and/or communication between the wireless devices 14 and the data acquisition system 16. The data acquisition system 16 wirelessly communicates with the wireless devices 14. The data acquisition system 16 could communicate with the wireless devices 14 proximate to the structure 12 (e.g., a hand-held reader/writer) or distant from the wireless devices (e.g., at a central data processing station).

The data acquisition system 16 typically includes a processor or similar computing device operating under the control of software so that data acquired by the sensors 20 may be analyzed to characterize any defects in the structure (see FIG. 4, block 30). The processor could be embodied by a computer such as a desktop, laptop, tablet computer, or portable processing device capable of processing the data generated by the wireless devices 14. For example, the data acquisition system 16 could be a hand-held reader/writer that a technician could use to scan the structure 12 proximate to the wireless devices 14 and download data acquired by the wireless devices during on-the-ground inspection. In addition, the hand-held reader/writer could be employed intermediately to collect or log the data from the wireless devices 14 such that the data acquisition system 16 could then download the data from the hand-held reader for further processing. Similarly, the data acquisition system 16 could create a database to store the data acquired by the wireless devices 14 in response to the data collected by the data acquisition system.

Furthermore, the data acquisition system 16 is capable of interrogating each wireless device 16. For instance, the data acquisition system 16 may include a pulser/receiver card, or similar device, that is utilized to interrogate the wireless devices 14 such that the wireless devices are capable of transmitting signals within and receiving signals from the structure 12, such as ultrasonic stress waves. Similarly, the wireless device 14 may be employed to interrogate itself. For example, the wireless device 20 could generate a pulse within the structure 12 that is translated into a signal indicative of the structure when received back at the sensor. Thus, the sensor 20 could be active and generate the pulse, or the sensor could be powered by an external source to enable the sensor to generate a pulse signal within the structure 12. In one embodiment of the present invention, NDI sensors, such as piezo-electric sensors, can act as passive ultrasonic receivers, where a received stress pulse is translated into an electric pulse. In the passive mode, the sensors could collect stress wave data that emits an electric pulse that travels through an IC chip and is stored as digital data on the RFID tag. Also, the piezo-electric sensors could act as active transmitters that emit a voltage pulse that is translated into a stress wave, transmitted within the structure 12, and returned to the sensors with data indicative of the structure.

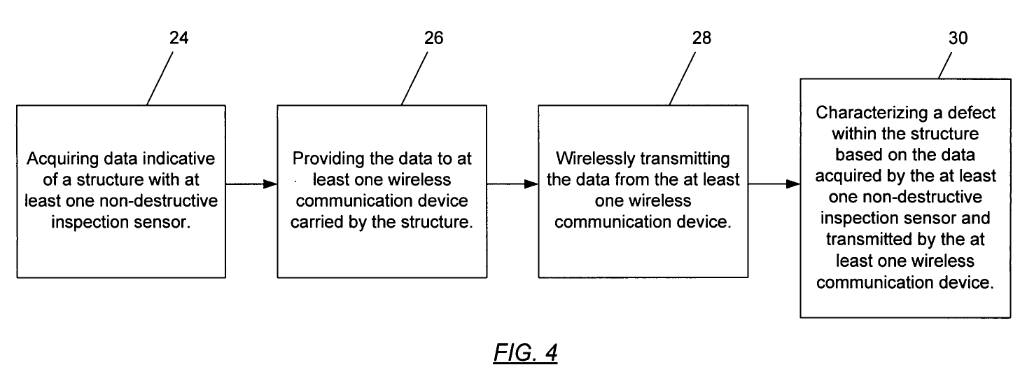

Each wireless device 14 is typically in communication with the data acquisition system 16, either directly or via a network, to process the data accumulated by the wireless devices. In further embodiments of the present invention the data acquisition system 16 may interrogate the wireless devices periodically or continuously and may even be used to process data while the structure 12, such as an aircraft, is in use (e.g., in flight). Thus, a data acquisition system 16 could be employed for on-aircraft monitoring of specific locations on the aircraft that are susceptible to the formation of defects, such as that shown in FIG. 3. The data acquisition system 16 could also interrogate or download data from the wireless devices 14 individually, in specific patterns, or simultaneously. Furthermore, each wireless device 14 could include an identifier such that the acquired data may be associated with a specific wireless device.

The wireless devices 14 are capable of storing data indicative of the structure 12. In particular, the wireless devices 14 may store the data as it is acquired by the wireless devices, or the wireless devices may store information provided by the data acquisition system 16. Thus, the data acquisition system 16 may write data to the wireless devices 14 such that the wireless devices may store processed information from the data acquisition system 16, which may be accessed and used at a later time for further analysis. Moreover, the wireless devices 14 may not only store data acquired by the wireless devices and information provided by the data acquisition system 16, but also additional data, such as information relating to the operating environment (e.g., temperature), maintenance (e.g., maintenance schedule or procedures), and/or specific characteristics of the structure 12 (e.g., specifications). The wireless devices 14 could be reset, i.e., the data erased, after the data is communicated to the data acquisition system 16, periodically, or at any other desired time. It is also understood that each wireless device 14 could include a processor for processing the NDI data and generating information indicative of a defect in the structure 12. In this regard, the wireless device 14 could then transfer the data to a data acquisition system 16 or other device to be displayed or analyzed to determine if remedial action is required. However, the data acquisition system 16 will typically perform some or all of the processes associated with analyzing the data acquired by the wireless devices 14.

The data acquisition system 16 generates information indicative of the structure 12, including, for example, at least those defects detected within the structure, based on data acquired by the wireless devices 14 and may display an image, such as an A-scan, a B-scan, or a C-scan. The data acquisition system 16 is capable of generating information indicative of a defect and may also allow a user to store and edit previously created images. Therefore, a permanent record of the images may be kept for future use or record keeping. However, it is understood that in addition to displaying images with a display, the data acquisition system 16 could mathematically collect and analyze data from the wireless devices 14 that a technician could use to characterize a defect based on the data. Based on the characterization of the defect, a technician may make a decision whether to repair, replace, or take other action to address the defect. Software for analyzing the data acquired by the wireless devices 14, as known to those of ordinary skill in the art, is typically used to generate information characterizing defects in the structure 12. Thus, the data stored by the wireless sensors 14 is generally in a format that may be employed with software for analyzing the data, as well as in a format capable of being processed by the data acquisition system 16.

Embodiments of the present invention provide several advantages. For example, the wireless devices 14 are capable of being located at various locations on a structure 12, including areas that are typically not easily accessed by conventional inspection systems. In addition, the wireless devices 14 do not require wiring or external power sources such that the wireless devices may be easily and efficiently monitored for improved structural health assessment. Moreover, if the structure 12 is an aircraft, the wireless devices 14 may save weight, installation, and maintenance costs. The wireless devices 14 are also thin and may be attached to a variety of structures 12 such that the wireless devices are adaptable for inspecting any number of structures. The wireless sensors 14 are also capable of acquiring and storing data indicative of the structure 12, as well as other data related to the structure. Furthermore, the data acquisition system 16 may process the data acquired by the wireless devices 14 and provide information regarding a variety of defects, such as the type and location of the defect in the structure 12.

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A system for assessing and monitoring a structure comprising:
at least one radio frequency identification tag; and
at least one non-destructive inspection sensor for receiving a signal following propagation through at least a portion of the structure and acquiring data indicative of a defect in the structure based on the signal and providing the acquired data to the radio frequency identification tag.

2. The system according to claim 1, wherein the radio frequency identification tag is integrated with the non-destructive inspection sensor.

3. The system according to claim 1, wherein the non-destructive inspection sensor comprises a piezoelectric sensor.

4. The system according to claim 1, wherein the radio frequency identification tag and non-destructive inspection sensor are attached to, or embedded within, the structure.

5. The system according to claim 1, wherein the radio frequency identification tag and non-destructive inspection sensor are carried by an appliqué or a repair patch.

6. The system according to claim 1, wherein the radio frequency identification tag and non-destructive inspection sensor are capable of storing the data acquired by the non-destructive inspection sensor.

7. The system according to claim 1, further comprising a a high permeability material backing positioned between the radio frequency identification tag and the structure.

8. The system according to claim 7, wherein the high permeability material backing is positioned adjacent to a metallic structure.

9. The system according to claim 1, further comprising a data acquisition system capable of wirelessly communicating with the radio frequency identification tag and providing information indicative of a defect in the structure based on the data acquired by the non-destructive inspection sensor.

10. The system according to claim 9, wherein the radio frequency identification tag and non-destructive inspection sensor are capable of storing information provided by the data acquisition system.

11. The system according to claim 1, further comprising at least one power source for powering at least one of the radio frequency identification tag and the non-destructive inspection sensor.

12. A method for assessing and monitoring a structure comprising:
transmitting a signal within the structure;
detecting the signal with at least one non-destructive inspection sensor following propagation through at least a portion of the structure;
acquiring data indicative of a defect in the structure with the at least one non-destructive inspection sensor based on the detected signal;
providing the data to at least one wireless communication device carried by the structure;
wirelessly transmitting the data from the at least one wireless communication device; and
characterizing a defect within the structure based on the data acquired by the at least one non-destructive inspection sensor and transmitted by the at least one wireless communication device.

13. The method according to claim 12, further comprising positioning the at least one wireless communication device and non-destructive inspection sensor within or adjacent to the structure.

14. The method according to claim 13, wherein positioning comprises embedding the at least one wireless communication device and non-destructive inspection sensor within the structure.

15. The method according to claim 13, wherein positioning comprises positioning the at least one wireless communication device and non-destructive inspection sensor within an appliqué or a repair patch.

16. The method according to claim 12, further comprising positioning a high permeability backing material between the at least one wireless communication device and the structure.

17. The method according to claim 12, further comprising storing the acquired data with the at least one wireless communication device or non-destructive inspection sensor.

18. The method according to claim 12, further comprising storing information indicative of the defect with the at least one wireless communication device or non-destructive inspection sensor.

19. A wireless device for assessing and monitoring a structure comprising:
at least one radio frequency identification tag capable of acquiring data indicative of the structure, wherein the radio frequency identification tag is also capable of wirelessly communicating with a data acquisition system.

20. The wireless device according to claim 19, wherein the radio frequency identification tag is attached to, or embedded within, the structure.

21. The wireless device according to claim 19, wherein the radio frequency identification tag is carried by an appliqué or a repair patch.

22. The wireless device according to claim 19, wherein the radio frequency identification tag is capable of storing the acquired data, and wherein the radio frequency identification tag is capable of transmitting the acquired data to the data acquisition system.

23. The wireless device according to claim 19, wherein at least one characteristic of the radio frequency identification tag is modified to acquire data indicative of the structure.

24. The wireless device according to claim 23, wherein the characteristic comprises a deformed antenna.

25. The method of claim 12, wherein transmitting comprises transmitting signals within the structure with at least one of the non-destructive inspection sensor, a data acquisition system, an impact to the structure, and a pulser card.

26. The method of claim 12, wherein transmitting comprises transmitting stress waves within the structure.
